(19) United States
(12) Patent Application Publication
(10) Pub. No.: US 2026/0309632 A1
(43) Pub. Date: Oct. 8, 2026

(54) CARTRIDGE PROVIDING REAL-TIME COLORIMETRIC DETECTION AND/OR TURBIDIMETRIC DETECTION OF MICROORGANISMS VIA IMAGE CAPTURE ON A MOBILE DEVICE

(71) Applicant: SEETHRU DIAGNOSTICS, LLC, Wildwood, MO (US)

(72) Inventor: Andrew P. TOMARAS, Wildwood, MO (US)

(21) Appl. No.: 19/481,596

(22) PCT Filed: May 8, 2024
(86) PCT No.: PCT/US2024/028284

§ 371 Date: Nov. 5, 2025

Related U.S. Application Data
(60) Provisional application No. 63/501,255, filed on May 10, 2023.

(51) Int. Cl. G01N 21/78; G01N 21/01; G01N 21/59

(52) U.S. Cl. CPC G01N 21/78; G01N 21/01; G01N 21/59

(57) ABSTRACT

The present disclosure is a cartridge comprises a main body having a plurality of chambers for receiving the fluid sample. Each of the plurality of chambers includes a background dark contrast pattern that becomes hazy in response to a turbidity change in the sample caused by growth of the microorganism. A color change in the sample may also occur. At least one of the plurality of chambers including a nutrient-rich material to facilitate the microorganism growth or a chemical dye that changes a color of the fluid sample in response to the microorganism growth. The cartridge can be used for detecting the presence of a microorganism, for characterizing a microorganism, and/or for identifying a microorganism in a fluid sample.

### CROSS-REFERENCE TO RELATED APPLICATIONS

This application claims priority to U.S. Provisional Patent Application Ser. No. 63/501,255, filed May 10, 2023 and entitled “Cartridge Providing Real-Time Colorimetric Detection and/or Turbidimetric Detection of Microorganisms via Image Capture on a Mobile Device,” which is herein incorporated by reference in its entirety.

### COPYRIGHT

A portion of the disclosure of this patent document may contain material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention generally relates to the detection of microorganisms in samples and, specifically, to detecting the presence or absence of microorganisms from a sample using colorimetric-based detection and/or turbidimetric-based detection via image capture on a mobile device (i.e., cell phone, tablet, etc.) and an app that utilizes a computer vision/image processing detection algorithm.

### BACKGROUND OF THE INVENTION

Microorganisms are ubiquitous in nature. They are often located on and in mammals. In many cases, their presence is benign or even beneficial to their hosts. In some cases, however, microorganism presence can lead to environmental contamination and/or direct host illness. Thus, the expedited detection of their presence or absence is crucial to avoiding widespread illness. Expedited detection of their presence or absence is also important to prevent food and water shortages. Unfortunately, detecting microorganisms from clinical, agricultural, or environmental sites has historically been limited to sophisticated laboratories with lengthy turnaround times (TATs) with few or no viable solutions to expeditiously detect and characterize microorganisms in the field.

For example, there are many applications in the field of analytical research and clinical testing that utilize complex methods for analyzing liquid samples. Among those methods are optical measurements that measure absorbance, fluorescence, luminescence, or optical scattering measurements. Optical laser scattering is one of the most sensitive methods, but its implementation can be very challenging, especially when analyzing biological samples in which suspended particles are relatively transparent in the medium. In addition, preparing and measuring biological samples can be costly and time-consuming due to the need to ensure a sufficient volume of the sample or of any unknown microorganisms within the sample. Similarly, detection methods involving the use of absorbance, fluorescence, and/or luminescence all require the use of sophisticated instrumentation and expensive reagents that typically require specialized storage conditions (e.g., under refrigeration or in a freezer). Additionally, instrument operation and interpretation of results often requires prior training and proficiency for each individual platform. Thus, it is impractical to use these more complex systems in a remote location where a fluid sample must be tested. Consequently, contaminated food or water or early stages of mammalian infection can go undetected, allowing for microorganism replication and downstream consequences with contamination and/or infection.

Accordingly, there is a need for improved devices, systems, and methods that can quickly detect, identify, and provide at least a preliminary characterization of microorganisms present in a sample. The proposed invention is intended to provide onsite (e.g., home-based) capabilities for microorganism detection and/or characterization without the need for expensive equipment or technically challenging instructions for use. All these and other objects of the present invention will be understood through the detailed description of the invention below.

### SUMMARY OF THE INVENTION

In one aspect, the present invention is a cartridge having chambers for holding a sample and wherein the cartridge provides colorimetric-based detection and/or turbidimetric-based detection of microorganisms in the sample, as described relative to FIGS. 1-6.

In another aspect, the present invention is a system with a cartridge and an App on a mobile device for colorimetric-based detection and/or turbidimetric-based detection of microorganisms in a sample via image capture on the mobile device, as described relative to FIGS. 1-6.

In a further aspect, the present invention is a cartridge for detecting the presence of a microorganism, for characterizing a microorganism, and/or for identifying a microorganism in a fluid sample. The cartridge comprises a main body having a plurality of walls that define a plurality of chambers for receiving the fluid sample. The main body further including an optically clear top surface for each of the plurality of chambers. The plurality of walls includes a lower wall at the bottom of each of the plurality of chamber. A background contrast pattern being on or below the lower wall and can be viewed through the optically clear top surface. The background contrast pattern becomes hazy, after a time period, in response to a turbidity change in the fluid sample caused by growth of the microorganism. At least one of the plurality of chambers including a nutrient-rich material to facilitate the microorganism growth.

In another aspect, the present invention is a cartridge for detecting the presence of a microorganism, for characterizing a microorganism, and/or for identifying a microorganism in a fluid sample. The cartridge comprises a main body having a plurality of walls that define a plurality of chambers for receiving the fluid sample. The main body further includes an optically clear top surface for each of the plurality of chambers. The plurality of walls include a lower wall at the bottom of each of the plurality of chamber. A background contrast pattern being on or below the lower wall and can be viewed through the optically clear top surface. The background contrast pattern becomes hazy, after a time period, in response to a turbidity change in the fluid sample caused by growth of the microorganism. At least one of the plurality of chambers including a nutrient-rich material to facilitate the microorganism growth. And at least one of the plurality of chambers including a chemical dye that changes a color of the fluid sample in response to the microorganism growth.

In a further embodiment, the present invention is a cartridge for detecting the presence of a microorganism, for characterizing a microorganism, and/or for identifying a microorganism in a fluid sample. The cartridge comprises a main body having a plurality of walls that define a plurality of chambers for receiving the fluid sample. The main body further includes an optically clear top surface for each of the plurality of chambers. The plurality of walls include a lower wall at the bottom of each of the plurality of chamber. A background contrast pattern being on or below the lower wall and can be viewed through the optically clear top surface. The background contrast pattern becomes hazy, after a time period, in response to a turbidity change in the fluid sample caused by growth of the microorganism. At least one of the plurality of chambers includes a chemical dye that changes a color of the fluid sample in response to the microorganism growth.

In another embodiment, the present invention is a cartridge for detecting the presence of a microorganism, for characterizing a microorganism, and/or for identifying a microorganism in a fluid sample. The cartridge comprises a main body having a plurality of chambers for receiving the fluid sample. Each of the plurality of chambers includes a background contrast pattern that becomes hazy in response to a turbidity change in the sample caused by growth of the microorganism. At least one of the plurality of chambers including a nutrient-rich material to facilitate the microorganism growth or a chemical dye that changes a color of the fluid sample in response to the microorganism growth.

In yet another embodiment, the present invention is a cartridge for detecting the presence of a microorganism, for characterizing a microorganism, and/or for identifying a microorganism in a fluid sample. The cartridge comprises a main body having a plurality of chambers for receiving the fluid sample. Each chamber includes a background contrast pattern that can be viewed from outside the main body. The background contrast pattern becomes hazy, after a time period, in response to a turbidity change in the fluid sample caused by growth of the microorganism.

In each of the various embodiments, the cartridge may further include a heating element that raises the temperature in the chambers to increase a growth rate of the microorganism.

In another aspect, the present invention is also the cartridge from any of the previous embodiments in combination with an App capable of being operated on a mobile device. The App assists in taking images of the chambers.

In a further aspect, the present invention is also the cartridge from any of the previous embodiments in combination with an App capable of being operated on a mobile device. The App assists in confirming the haziness to the background contrast pattern.

In yet a further aspect, the present invention is also the cartridge from any of the previous embodiments in combination with an App capable of being operated on a mobile device. The App assists in confirming the change in color of the sample.

In yet another aspect, a method of detecting the presence of a microorganism in a sample comprises using a cartridge from any of the previous embodiments to receive the sample and determine the presence of a microorganism.

In another aspect, a method of identifying the type or class of a microorganism in a sample comprises using a cartridge from any of the previous embodiments to receive the sample and identify the microorganism.

In a further aspect, a method for characterizing a microorganism in a sample comprises using a cartridge from any of the previous embodiments to receive the sample and characterize the microorganism.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

The present invention will be described with greater specificity and clarity with reference to the following drawings, in which:

FIG. 1A illustrates a top view of one exemplary embodiment of a cartridge according to the one embodiment of the present disclosure.

FIG. 1B illustrates a side perspective view of the cartridge of FIG. 1A.

FIG. 2 illustrates a series of images from one of the chambers in the cartridge.

FIG. 3 illustrates images from three chambers in the cartridge in which there are additives in two chambers and no additives in a control chamber.

FIG. 4 schematically illustrates an alternative cartridge having multiple chambers.

FIG. 5A illustrates a mobile device capturing an image of one of the chambers of the cartridge.

FIG. 5B illustrates the display screen of the mobile device after capturing the image of one of the chambers of the cartridge in FIG. 5A.

FIG. 6A illustrates the display screen of the mobile device in which the App is providing the user with instructions for use.

FIG. 6B schematically illustrates the display screen of the mobile device showing the various functions for the App when used in conjunction with the cartridge.

While the invention is susceptible to various modifications and alternative forms, specific embodiments will be shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE DRAWINGS

The drawings will herein be described in detail with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

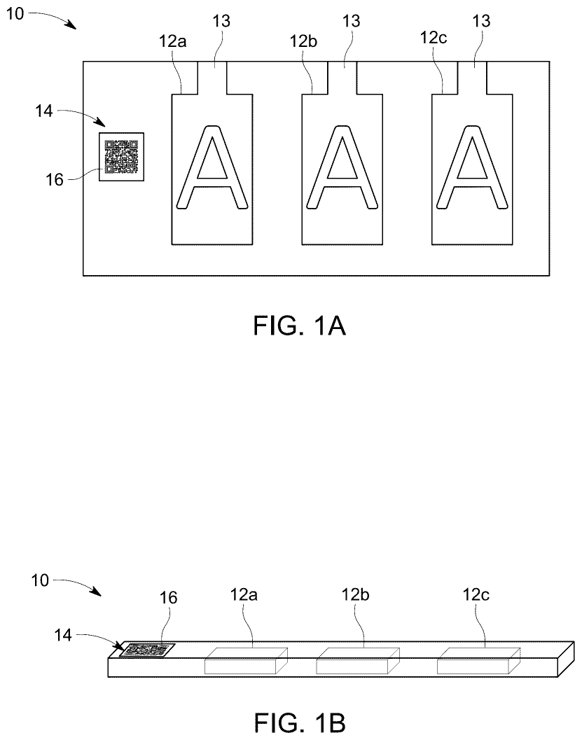

FIG. 1 schematically illustrates a disposable cartridge 10 with three chambers 12a, 12b, 12c that enable real-time turbidimetric detection and/or colorimetric turbidimetric of microorganisms in a sample. The three chambers 12 allow for concurrent testing of the sample in the presence of two different broth additives in two chambers 12a, 12b and with no additive in a third chamber 12c, which serves as a “control.” In the embodiment of FIG. 1, the cartridge 10 has a length of about 80 mm, a width of about 48 mm, and a thickness of about 8 mm. Each chamber 12 is about 12 mm, by about 32 mm, by about 4 mm. The spacing between chambers 12 is about 10 mm. Other shapes and sizes for the cartridge 10 and chambers 12 will equally work.

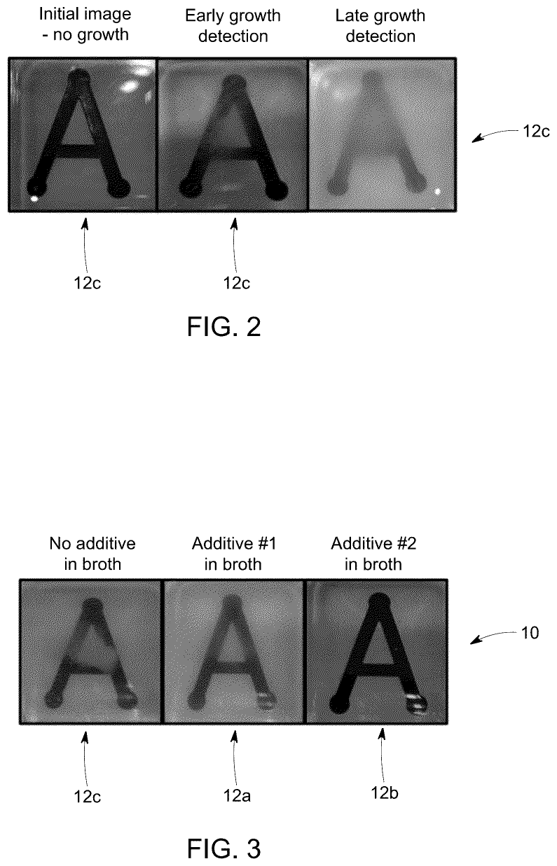

In some embodiments, the main body of the cartridge 10 includes a plurality of walls that are preferably constructed of transparent plastic to allow for image capture via a camera of a mobile device. Alternatively, at least the top surface on each of the three chambers 12 is made of a transparent material. The lower wall defining the bottom surface of the cartridge 10 includes a dark contrasting pattern, as exemplified by the black capital letter “A”, which is used for turbidity detection in samples with microbial growth, as shown in FIGS. 2 and 3 below. The dark contrasting pattern can be on the outside surface of the cartridge 10 (e.g., below the cartridge 10 and integral with the cartridge 10, or below the cartridge 10 a separate component from the cartridge 10). The dark contrasting pattern can be a white surface or structure (or light-colored surface or structure) with the darker contrasting pattern (e.g., the letter “A”) printed thereon. Alternatively, the lower wall of the cartridge 10 itself can also have a distinct color (e.g., white) with the “A” thereon. In each of these variations, the dark contrasting pattern enhances the visualization of any colorimetric changes caused by a chemical dye (e.g., pH-changing dye) in the chamber 12 when the microorganisms grow. In some embodiments, the individual chambers 12 of the cartridge 10 will each have a loading capacity of 1-10 milliliters of fluid, with individual loading ports or holes 13 present at the top of each chamber 12. Preferably, each of the loading ports 13 is readily opened and closed and can be sealed.

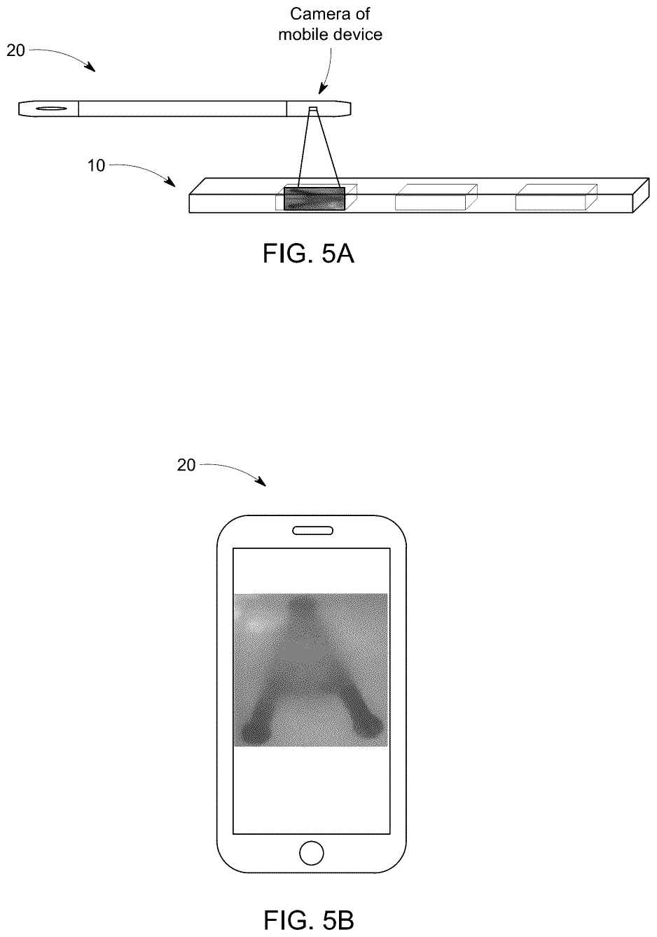

The cartridge 10 includes a solid support surface 14 that includes the cartridge serial number and/or test registration features that is typically identified via a code 16 (e.g., two-dimensional barcode or a QR code) or other accompanying sample information (i.e., sample ID, date/time of sample collection, other testing outputs, etc.). When the code 16 is scanned by a camera of a mobile device, it provides various pieces of information about the cartridge 10 and the testing. The code 16 may provide information on the type of test being requested for initiation, verification that the cartridge 10 itself has not exceeded its predetermined shelf life (e.g., past its expiration date), and to provide the App with a consistent image that can serve to ensure an appropriate picture is being captured (e.g., not out of focus, too close/far). In other words, as shown in FIGS. 5a and 5b, the code 16 (or other reference identifier, such as a symbol, crossing lines, geometric shape, etc.) serves as a reference point for maintaining the proper distance and registration between the cartridge 10 and an image capture device, such as a mobile phone 20. Other types of codes 16 (bar codes) or simple alphanumeric characters may be used as well.

FIG. 2 illustrates three exemplary images of a single chamber 12 (e.g., chamber 12c) of the cartridge 10 that contains a liquid sample with viable microorganisms. The three images in FIG. 2 are taken over a time period at t=0, t=180 min, and t=240 min. As can be seen, the turbidimetric characteristics and the colorimetric characteristics in the fluid sample change due to the present of a viable microorganism. In the initial image, the color of the clamber 12c is a greenish grey due to the sample mixture with Nutrient Broth (which is brownish green) and bromothymol blue. In the second image, showing the early growth at 180 minutes, the color is a browner green with some yellow highlights. In the third image, showing the late growth at 240 minutes, the color is an orangish yellow due to the microorganisms metabolizing the media, thereby creating a high pH in the sample. The third image shows turbidimetric changes, creating the burring or hazy “A”, as well as colorimetric changes. In one embodiment, the turbidimetric changes are more initially perceived, followed by more pronounced colorimetric changes at a later point in time.

In this example of FIG. 2, the cartridge 10 includes a clear top and bottom and the underlying background is a white paper located on the outside surface of the cartridge 10. The letter “A” is located on the white paper. The actual colors of the chamber 12c can be seen in U.S. Provisional Application Ser. No. 63/501,255, filed May 10, 2023, and entitled “Cartridge Providing Real-Time Colorimetric Detection and/or Turbidimetric Detection of Microorganisms via Image Capture on a Mobile Device,” which is herein incorporated by reference in its entirety. Other types of color changes are contemplated as well. By detection of these turbidimetric characteristics and colorimetric characteristics via image capture and algorithmic processing via a mobile device, the presence of the microorganism is determined. The image capture is aided by a background dark contrast pattern, which in the illustrated embodiment is shown as the capital letter “A” on a white paper.

To provide for the changing turbidimetric characteristics and the colorimetric characteristics, the chamber 12 of the cartridge 10 includes nutrient-rich broth to facilitate the expedited replication and growth of the microorganism to enable macroscopic detection over time. In some embodiments, the broth will also contain a chemical dye, such as bromothymol blue, that will change color as the microorganisms metabolize the broth and alter the pH of the entire sample. Collectively, the change in broth color and the emergence of turbidity will be detected over the dark contrast pattern in the background of all images captured, as shown in FIG. 2. Other examples of a chemical dye include, but are not limited to, bromocresol purple, resazurin, and propyl red. Examples of the nutrient-rich broth include, but are not limited to, Nutrient Broth, 2XYT Broth, and NZCYM Broth.

FIG. 3 illustrates an alternative embodiment of the cartridge 10 wherein the broth is supplemented with biological/chemical additives that will selectively permit/inhibit the growth of different microorganisms. FIG. 3 illustrates three different chambers 12 (e.g., 12a, 12b, and 12c) of the same cartridge 10 in which the images of a microorganism-containing sample show differential growth patterns in the presence/absence of a selective biochemical additive. The three chambers 12, the color of the clambers are initially a greenish gray due to the sample mixture with and bromothymol blue and Nutrient Broth. All images were captured at the same point in time using an iphone 11 with default camera images. The first chamber 12c contains no additive (a “control”), such that the microorganism is free to grow. The second chamber 12a contains Additive #1 (Bacteriophage #1) that only slightly affects the growth of the microorganism. The third chamber 12b contains Additive #2 (Bacteriophage #2) that significantly affects the growth of the microorganism, such that the Additive #2 has effectively killed the microorganism. All three images were captured at the same time (t=240 min) after the same sample was placed in the three chambers 12a, 12b, 12c. The first image of the chamber 12c has changed colors from a greenish gray to a yellowish brown and the “A” is blurred due to turbidity. The second image of the chamber 12a has changed colors from a greenish gray to a lighter yellowish brown and the “A” is slightly blurred. The third image of the chamber 12 b has maintained the greenish gray color over the 240 minutes and the “A” is clear because the Additive #2 has, in essence, killed the microorganism or at least substantially limited its growth, preventing any detectable turbidity change or color change. The actual colors of this chamber 12 c can be seen in U.S. Provisional Application Ser. No. 63/501,255, filed May 10, 2023, and entitled “Cartridge Providing Real-Time Colorimetric Detection and/or Turbidimetric Detection of Microorganisms via Image Capture on a Mobile Device,” which is herein incorporated by reference in its entirety.

The incorporation of different types of additives in different chambers 12 will aid in the characterization of microorganisms (or class of microorganisms) present in the sample, which will provide additional information to the user. For instance, inclusion of an effective concentration of an antibiotic (or other antimicrobial agent) can help to discern the antibiotic resistance/susceptibility profile of any microorganisms input into the cartridge, with simultaneous determination of general microorganism presence/absence (in the antibiotic-free chamber) and antimicrobial resistance/susceptibility (in the antibiotic-containing chamber). Similarly, by preloading reagents that can assist in the identification of the microorganism's genus and species, this profile parameter can also be ascertained in parallel. As used herein, “identifying” (or the “identification” of) the microorganism is intended to mean the determination of the organism's genus and/or species (i.e., Escherichia coli). As used herein, “characterizing” (or the “characterization” of) the microorganism is intended to mean the determination of various behavioral attributes of any microorganisms present in the cartridge, such as antimicrobial resistance/susceptibility patterns.

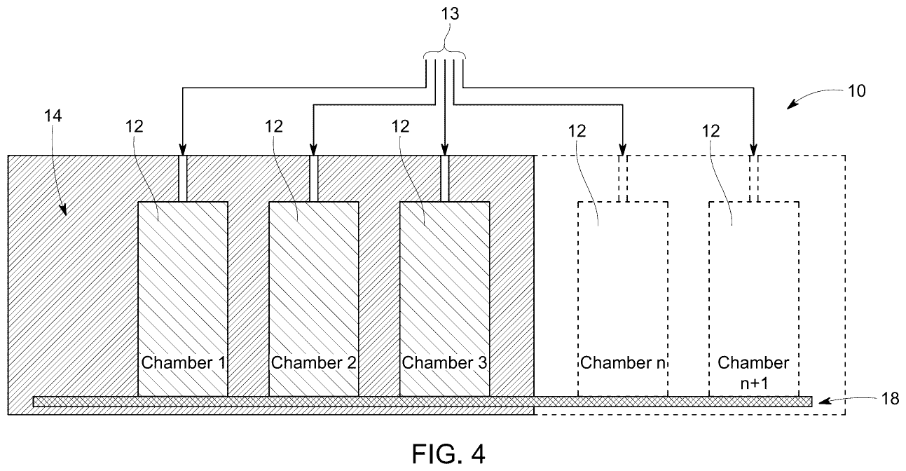

While only two chambers 12 with two additives are illustrated in FIG. 3, the present invention contemplates more chambers with different additives (see FIG. 4), such as (i) four chambers where three chambers have three different additives, (ii) five chambers where four chambers have four different additives, and (iii) (i) six chambers where five chambers have five different additives. Known microorganisms will create a certain “pattern” of color and turbidity in the plurality of chambers such that a specific microorganism will be easily identified and/or characterized. Examples of biological/chemical additives include the aforementioned antibiotics agents, antimicrobial agents, bacteriophages, and nutritional additives that selectively enrich the growth of specific microorganisms.

FIG. 4 schematically illustrates an alternative cartridge 10 with multiple chambers 12. When the cartridge 10 is used with multiple additives and multiple dyes, more chambers 12 are needed. As shown in the first three chambers 12 of the cartridge 10, the dark contrast patterns are diagonal lines at the bottom of or below the chambers that are visible through the optically transparent top surface. Other symbols (e.g., #, &, @, etc.) and/or alphameric characters can be used as well for the dark contrast pattern. In a further embodiment, the dark contrast pattern is a first color and the anticipated change in broth color is to a second color, whereby the first and second colors cause the underlying dark contrast pattern to be a third color. For example, the dark contrast pattern is a blue and the anticipated change in broth color is to yellow in the presence of a certain microorganism, such that, after a certain period, the underlying dark contrast pattern appears green (and hazier) when the certain microorganism is present.

In some embodiments, as shown in FIG. 4, a portable heating element 18 (e.g., a disposable or rechargeable hand warmer) is used to increase the temperature of the chambers 12 to levels that support optimal microorganism growth rates, thereby expediting the time-to-detection for various test offerings. In one embodiment, the cartridge 10 is laid flat on the surface of the portable heating element 18, maximizing contact and heat transfer to the incubating sample(s) in the chambers 12. In other embodiments, the cartridge 10 includes recess(es) into which the portable heating elements 18 are placed. Further, a thermocouple may be coupled to the cartridge 10 at a certain location to ensure the proper temperature range is maintained by the heating element 18.

FIGS. 5A and 5B schematically illustrate a mobile device 20 that includes a camera for capturing the image of a chamber 12 in the cartridge 10. The mobile device 20 (e.g., an iPhone) includes an App that works in conjunction with the cartridge 10 to help capture images, detect turbidity changes to the sample in the chamber 12 over a time period, and detect colorimetric changes to the sample in the chamber 12 over a time period.

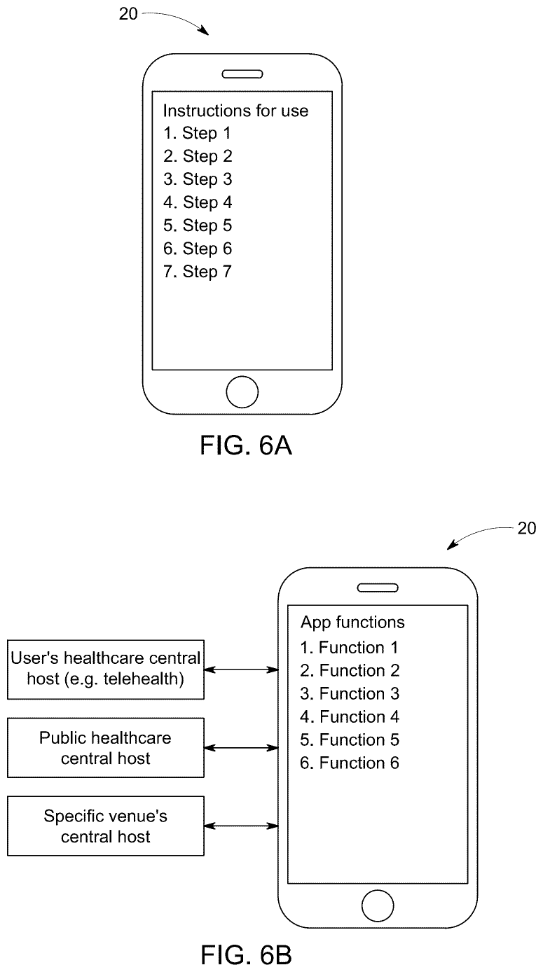

FIGS. 6A and 6B illustrate the mobile device 20 in which the display screen is providing information and functionality from the App. In FIG. 6A, the instructions for using the cartridge 20 are provided. The instructions include the following steps: (1) imaging the empty cartridge to register the new test and/or prompt test-specific instructions for collecting/loading the sample of interest; (2) verifying the proper loading of the new cartridge and establishing a baseline image at T=0; (3) prompting the user to collect images at a predefined, test-specific timepoint after test initiation (e.g., T=30 min; T=60 min; t=120 min); (4) prompting the user to collect images at a second predefined, test-specific timepoint after test initiation (e.g., T=180 min; T=240 min; t=300 min) (additional time-based image prompts may be included); (5) transmitting the images and information to a central host for determining the presence of microorganisms, identification of the microorganism, and/or characterization of the microorganism; (6) displaying standard patterns of color and/or turbidity as examples in which microorganisms were present, certain microorganisms were identified, and/or certain microorganisms were characterized; and (7) displaying information about dangers or treatment plans of certain microorganisms.

In one embodiment, the user can determine for himself or herself the presence of microorganisms, the identification of the microorganism, and/or the characterization of the microorganism. In another embodiment, images collected from the chambers 12 over time may be sent to a Cloud-based database for processing by a test-specific algorithm, after which results will be displayed and the user will be offered the option to send results to various, test-specific third parties (i.e., a tele-healthcare provider for infection testing; a public health department for water/food contamination, etc.). The microorganism growth is detected (and preferably identified or characterized) over a time period that is less than 8 hours, preferably less than 4 hours, more preferably less than 3 hours, and most preferably less than 2 hours, such as 60 minutes or 30 minutes.

FIG. 6B illustrates the various functions associated with the App on the mobile device 20. In one embodiment, the App or Apps are envisioned to perform one or more of the following functions: human/veterinary infection status (e.g., the presence of bacteria in the urine indicative of a urinary tract infection); detection of microorganism contamination in public/private drinking water supplies (i.e., E. coli presence requiring boil orders); detection of microorganism presence in processed meat/vegetation (i.e., E. coli contamination of spinach; Salmonella presence in uncooked chicken, etc.); detection of microorganisms in agricultural systems (i.e., S. aureus in dairy samples).

As such, in one embodiment, the inventive systems and methods provide for the detection of microorganisms in human urine (or the urine of other mammals). In another embodiment, the inventive systems and methods provide for the detection of microorganisms in human urine and the identification and/or characterization of the microorganisms in the human urine (or the urine of other mammals).

As such, in another embodiment, the inventive systems and methods provide for the detection of microorganisms in water, such as drinking water for humans, or water for farming or ranching functions. In another embodiment, the inventive systems and methods provide for the detection of microorganisms in such water and the identification and/or characterization of the microorganisms in the water.

In a further embodiment, the inventive systems and methods provide for the detection of microorganisms in processed meat/vegetation. In another embodiment, the inventive systems and methods provide for the detection of microorganisms in such processed meat/vegetation, and the identification and/or characterization of the microorganisms in the processed meat/vegetation.

In a further embodiment, the inventive systems and methods provide for the detection of microorganisms in dairy products (e.g., milk from a dairy cow). In another embodiment, the inventive systems and methods provide for the detection of microorganisms in such dairy products and the identification and/or characterization of the microorganisms in the dairy products.

These embodiments and obvious variations thereof are contemplated as falling within the spirit and scope of the claimed invention, which is set forth in the following claims. Moreover, the present concepts expressly include any and all combinations and subcombinations of the preceding elements and aspects.

1-10. (canceled)

11. A cartridge for detecting the presence of a microorganism, comprising:
a main body having a plurality of walls that define a plurality of chambers for receiving the fluid sample, the main body further including an optically clear top surface for each of the plurality of chambers, the plurality of walls including a lower wall at the bottom of each of the plurality of chamber,
a background contrast pattern associated with each of the lower walls that can be viewed through the corresponding optically clear top surface, the background contrast pattern becoming hazy when viewed through the optically clear top surface in response to a turbidity change in the fluid sample caused by growth of the microorganism;
at least one of the plurality of chambers including a nutrient-rich material to facilitate the microorganism growth; and
at least one of the plurality of chambers including a chemical dye, the chemical dye causing the fluid sample to change color in response to the microorganism growth; and
wherein the presence of the microorganism in the fluid sample is detected by the haziness of the background contrast pattern and/or the color change in the fluid sample in one or more of the chambers.

12. The cartridge of claim 11, wherein the optically clear top surface is a single piece of transparent plastic extending across the plurality of chambers.

13. The cartridge of claim 11, wherein the optically clear top surface is integrally formed with the main body, the lower walls are optically clear, the background contrast patterns being located outside the chambers and against the lower walls.

14. The cartridge of claim 11, wherein the optically clear top surface includes multiple pieces of transparent plastic, each of the multiple pieces of transparent plastic being associated with one of the plurality of chambers.

15. The cartridge of claim 11, wherein the at least one of the plurality of chambers that includes the nutrient-rich material also includes the chemical dye that changes a the color of the fluid sample in response to the microorganism growth.

16. The cartridge of claim 11, wherein the chemical dye changes the color of the fluid sample based on pH changes in the fluid sample caused by the microorganism growth.

17. (canceled)

18. The cartridge of claim 11, wherein the microorganism growth is detected over a time period that is less than 3 hours.

19. The cartridge of claim 11, wherein the haziness to the background contrast pattern and the color change is detected by an image captured from a mobile device.

20-33. (canceled)

34. The cartridge of claim 11, further including a heating element to raise the temperature in the plurality of chambers to increase the growth rate of the microorganism.

35. The cartridge of claim 11, further including a scannable code located on a surface of the cartridge to provide information on the testing for the microorganism using the cartridge.

36. A cartridge for detecting the presence of microorganisms in a fluid sample, comprising:
a main body having a plurality of walls that define a plurality of chambers for receiving the fluid sample, the plurality of chambers including at least a first chamber and a second chamber, the main body further including an optically clear top surface for each of the plurality of chambers, the plurality of walls including a lower wall at the bottom of each of the plurality of chambers;
a background contrast pattern associated with each of the lower walls of the plurality of chambers that can be viewed through the optically clear top surface;
a chemical dye located in each of the plurality of chambers; and
a first additive in the first chamber for inhibiting the growth of the microorganisms within the fluid sample, the second chamber being free of an additive that inhibits the growth of the microorganisms; and
wherein, after a time period, in the second chamber, (i) the background contrast pattern becomes hazy in response to a turbidity change in the fluid sample caused by the growth of the microorganisms, and (ii) the chemical die causes a color of the fluid sample to change to a first color in response to the growth of the microorganisms.
wherein, after the time period, in the first chamber (i) the background contrast pattern is noticeably less hazy than the background contrast pattern of the second chamber, and (ii) a color of the fluid sample is similar to an original color of the fluid sample due to the first additive inhibiting the growth of the microorganism.

37. The cartridge of claim 36, wherein the optically clear top surface is a single piece of transparent plastic extending across the plurality of chambers.

38. The cartridge of claim 36, wherein the optically clear top surface is integrally formed with the main body, the lower walls are optically clear, the background contrast patterns being located outside the chambers and against the lower walls.

39. The cartridge of claim 36, wherein the at least one of the plurality of chambers that includes a nutrient-rich material to facilitate the microorganism growth.

40. The cartridge of claim 36, wherein the chemical dye changes the color of the fluid sample based on pH changes in the sample caused by the microorganism.

41. The cartridge of claim 36, wherein the microorganism growth in the second chamber is detected by the haziness of the background contrast pattern and the color change.

42. The cartridge of claim 36, wherein the time period is less than 3 hours.

43. The cartridge of claim 36, further including a heating element to raise the temperature in the plurality of chambers to increase the growth rate of the microorganism.

44. The cartridge of claim 36, wherein the additive assists in identifying the microorganism.

45. The cartridge of claim 36, wherein images of the first and second chambers with the haziness of the background contrast pattern and change in color are captured with a mobile device that subsequently sends the images to a third-party health facility.
